(19) United States
(12) Patent Application Publication
(10) Pub. No.: US 2017/0010233 A1
(43) Pub. Date: Jan. 12, 2017

(54) METHOD FOR DETECTING WELD DEFECTS

(71) Applicant: ELECTRICITE DE FRANCE, Paris (FR)

(72) Inventor: Frederic Taillade, Clamart (FR)

(73) Assignee: ELECTRICITE DE FRANCE, Paris (FR)

(21) Appl. No.: 15/117,920

(22) PCT Filed: Feb. 12, 2015
(86) PCT No.: PCT/FR2015/050347

§ 371 Date: Aug. 10, 2016

(30) Foreign Application Priority Data
Feb. 12, 2014 (FR) 1451074

(51) Int. Cl. G01N 27/24; G01N 33/44

(52) U.S. Cl. CPC G01N 27/24; G01N 33/442

(57) ABSTRACT

A method of detecting a defect in a weld joining a first surface of a polyethylene-based material and a second surface of a polyethylene-based material, said method comprising the steps of: positioning a first electrode and a second electrode in proximity to the weld, so that at least one electric field line passes through said weld when a potential difference is applied between said first and second electrodes, measuring an electrical capacitance at a measurement frequency between said first electrode and said second electrode, the measurement frequency being higher than 65 MHz and lower than 1 GHz in order to compare the measured electrical capacitance to a reference value.

The present invention relates to a method of detecting a defect in a weld joining a first surface of a material based on polyethylene and a second surface of a material based on polyethylene.

The invention finds an application in many fields, such as those of nuclear or hydraulic power plants, and, more generally, in any industry where one has recourse to conduits based on polyethylene (PE), generally high-density polyethylene (HDPE), such as drinking water or natural gas systems, or else in certain power lines.

In fact, replacing existing pipelines, particularly steel pipelines, with PE conduits is known, which helps prevent problems with corrosion and steel pipeline maintenance, and helps strengthen the installations' resistance to earthquakes while reducing both costs and the time needed to change the conduits.

A PE conduit system is obtained by welding PE conduits by their ends, where a weld joins a first surface of a material based on polyethylene, for example an end of a first PE conduit, and a second surface of a material based on polyethylene, for example an end of a second PE conduit.

Consequently, the quality of the weld is paramount for a safe and sustainable use of such a system. However, depending on the conditions in which the weld is made, it is possible that the weld can be contaminated by impurities such as grease or dust or else that an air pocket can be locally created by a lack of fusion, which generates another type of defect, known as a cold fusion defect.

The quality of the weld can also be altered by its aging.

Consequently, controlling weld quality is essential for ensuring the proper operation of PE conduit systems. Therefore, there is a need for a method for detecting defects in such a weld.

A known method is based on the use of ultrasonic waves. Nevertheless, this method is limited to the detection of macroscopic defects, of a dimension on the order of several square millimeters, but does not enable smaller defects to be detected and, in particular, does not enable cold fusion defects to be detected.

The aim of the present invention is to remedy these disadvantages.

For this purpose, the aim of the invention is a method for detecting a defect in a weld joining a first surface of a material based on polyethylene and a second surface of a material based on polyethylene, said method comprising the steps of:
positioning a first electrode and a second electrode in proximity to the weld, so that at least one electric field line passes through said weld when a potential difference is applied between said first and second electrodes,
measuring an electrical capacitance at a measurement frequency between said first electrode and said second electrode, the measurement frequency being higher than 65 MHz and lower than 1 GHz in order to compare the measured electrical capacitance to a reference value.

Therefore, the method according to the present invention enables the detection of not only macroscopic defects, on the order of several square millimeters, but also enables defects that are tens of nanometers in size and cold fusion type defects to be detected.

In a particular embodiment, the measurement frequency is measured between 100 MHz and 300 MHz.

In this frequency range of operation, the electrical impedance measured between these two electrodes is similar as a first approximation to electrical capacitance.

In a particular embodiment, the detection method comprises a step of detecting a defect in the weld if, during the comparison step, the electrical capacitance measured is different by at least 1.5% from the reference value Cref.

In a particular embodiment, the reference value is on the order of 6 pF.

In a particular embodiment, the first surface belongs to a body presenting one thickness and the second surface belongs to a body presenting a thickness of the same order of magnitude as the thickness of the body of the first surface, and the predefined distance between the first electrode and the second electrode is within a range of values between a fifth of the value of said thickness and said thickness.

This value range ensures a measured electrical capacitance that is sufficiently different between a weld with a defect and a weld without a defect so that the defect is detected by the method.

Another aim of the invention is a capacitive probe for implementing the method as described previously, comprising a first electrode and a second electrode forming a capacitor presenting an electrical capacitance capable of depending on the presence of a defect in a weld joining a first surface of a polyethylene-based material and a second surface of a polyethylene-based material, and a device connected to said capacitor capable of measuring the capacitance value within a measurement frequency between 65 MHz and 1 GHz.

Such a probe is suitable for the detection method previously described.

In a particular embodiment, the device is an oscillation circuit, the resonance frequency measurement of which enables the measured electrical capacitance or a bridge circuit or an impedance meter or a vector network analyzer to be deduced.

In one particular embodiment, the measurement frequency is between 100 MHz and 300 MHz.

In one particular embodiment, the first electrode and the second electrode have a length on the order of a thickness of a body comprising said first surface and a body comprising said second surface.

In a particular embodiment, the first electrode is constituted of a coil of electrical wires.

Another aim of the invention is a use of a capacitive probe as described previously for detecting a defect in a weld joining a first surface of a polyethylene-based material and a second surface of a polyethylene-based material.

In a particular embodiment, the first surface is coaxial and adjacent to the second surface.

In a particular embodiment, the first surface is coaxial and at least partly fitted to the second surface.

Other characteristics and advantages of the invention will appear upon reading the following description. The description is purely illustrative and should be read in conjunction with the appended drawings, in which:

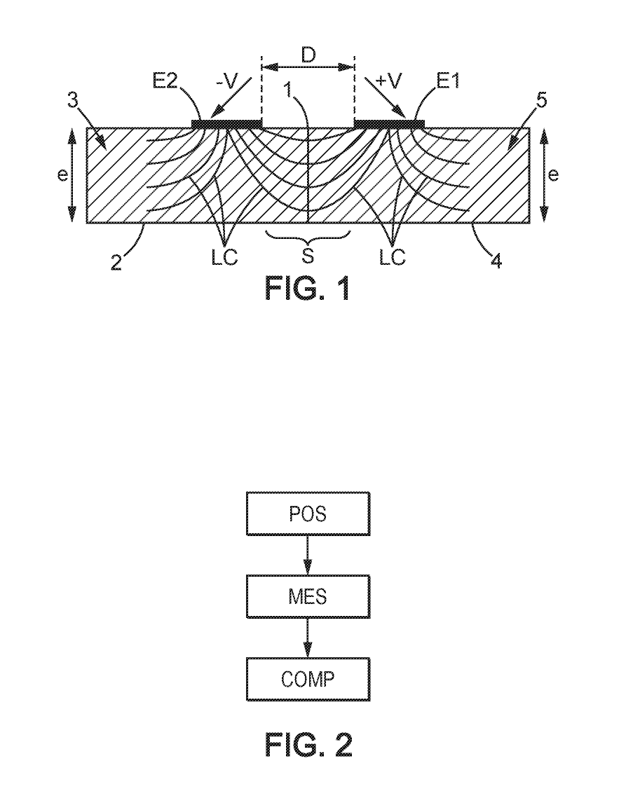

FIG. 1 is a longitudinal cross-sectional view of a detail of an installation of HDPE (high-density polyethylene) conduits to which a method of detecting defects according to the present invention is applied and on which first and second electrodes of a capacitive probe according to the present invention are placed;

FIG. 2 is a flow chart of the method for detecting defects according to the present invention;

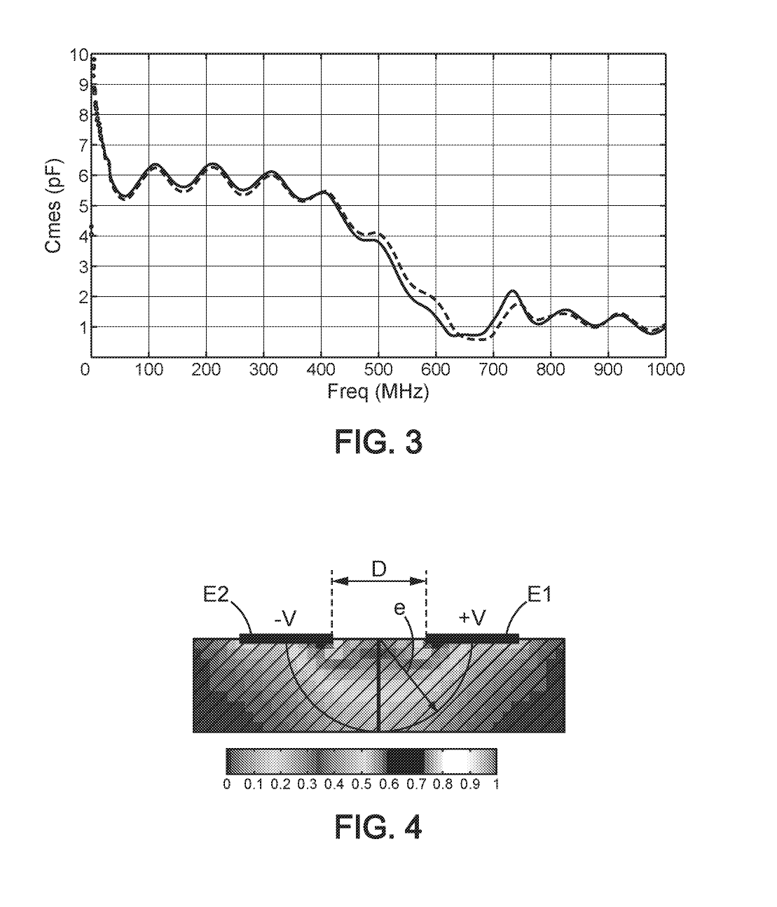

FIG. 3 is a diagram of the evolution of a measured electrical capacitance within a frequency range of operation;

FIG. 4 is a view of the detail from FIG. 1 illustrating the sensitivity of the capacitive probe of FIG. 2 according to the distance between the two electrodes;

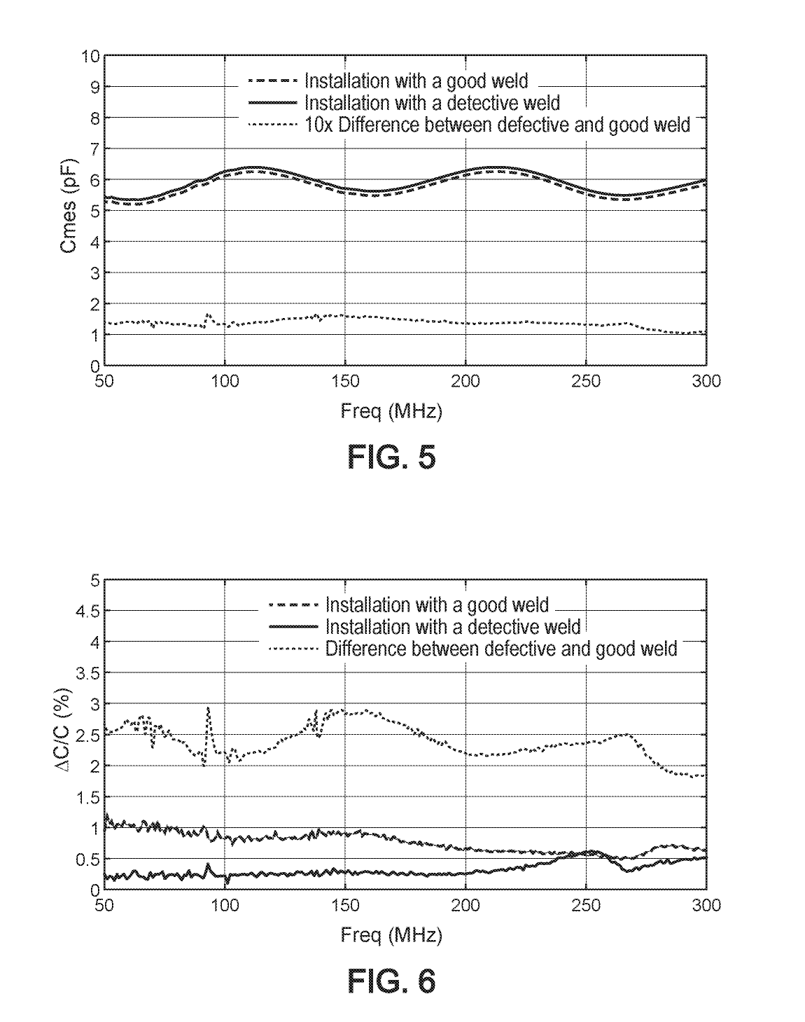

FIG. 5 is a diagram of the value of the measured electrical capacitance within a preferential frequency range of operation;

FIG. 6 is a diagram of the relative difference in the measured electrical capacitance from FIG. 5;

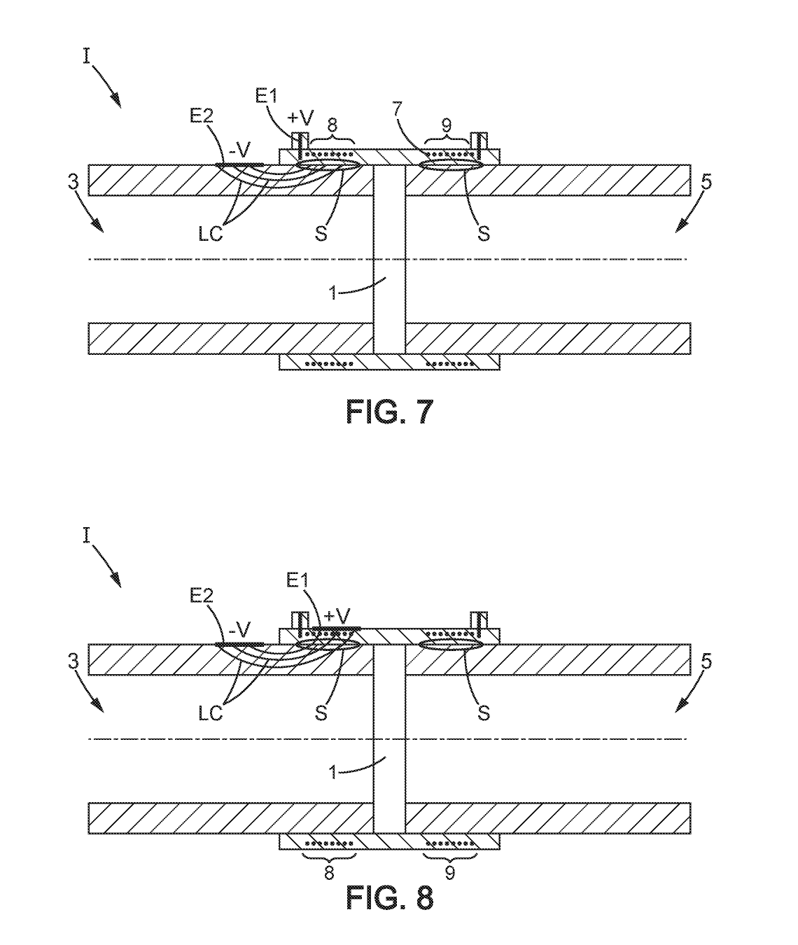

FIG. 7 is a longitudinal cross-sectional view of a detail from another installation of HDPE conduits to which the method for detecting defects according to the present invention is applied; and

FIG. 8 is a variation of FIG. 7.

The aim of the invention is a method for detecting a defect in a weld joining a first surface of a polyethylene (PE)-based material and a second surface of a polyethylene (PE)-based material.

The detection method according to the present invention is presented below in its application to an installation of high-density polyethylene (HDPE) conduits, this application of course not being limited to the scope of the invention.

Embodiment of the Method for Detecting Defects in an Installation of HDPE Conduits

In FIG. 1 is illustrated a detail of an installation of HDPE conduits, a weld S joining 1 two conduits, the first surface being a periphery surface 2 of a first conduit 3 and the second surface being a periphery surface 4 of a second conduit 5.

The first surface 2 and the second surface 4 present a general coaxial cylinder shape with a thickness e.

As seen in FIG. 1, weld S is deposited between two adjacent ends of conduits 3 and 5.

As seen in FIG. 1, a first electrode E1 is disposed on the outer side of the periphery surface 2 of the first conduit 3 and a second electrode E2 is disposed on the outer side of the periphery surface 4 of the second conduit 5.

Electrodes E1 and E2 are made from an electrically conductive material, for example a metal such as stainless steel. They preferably have identical shapes and dimensions.

Electrode E1 is at voltage +V, while electrode E2 is at voltage −V.

Due to the potential difference between electrodes E1 and E2, electric field lines LC are distributed between both electrodes, as represented in FIG. 1, some lines passing through weld S.

In these figures, the electrodes are represented in contact with external surfaces 2 and 4 of conduits 3 and 5. However, the invention is not limited to this embodiment.

It is possible for electrodes E1 and E2 to be disposed in contact with the inner surfaces of conduits 3 and 5.

It is also possible that the electrodes are not in contact with conduits 3 and 5 but, on the contrary, are disposed at a non-zero distance from the surfaces of conduits 3 and 5, either inside them, if conditions allow this, or outside them.

According to the invention, electrodes E1 and E2 must be disposed in proximity to weld S, i.e., electrodes E1 and E2 are disposed so that at least one field line LC passes through weld S.

The distance between electrodes E1 and E2 is noted D, and is chosen from a range of values, as indicated below.

Detection Method

The detection method comprises the steps of:
Positioning electrodes E1 and E2 in proximity to the weld S, as explained above. This step is referenced POS in FIG. 2, and
measuring an electrical capacitance Cmes at a measurement frequency between said first electrode E1 and said second electrode E2, the measurement frequency being higher than 65 MHz and lower than 1 GHz in order to compare the measured electrical capacitance to a reference value Cref. The measuring step is referenced MES in FIG. 2 and the comparison step is referenced COMP.

The measured electrical capacitance Cmes depends on the permittivity of the materials traversed by the field lines, which itself depends on the measurement frequency. In particular, electrical capacitance depends on the permittivity of the HDPE-based material of which conduits 3 and 5 are constituted, the permittivity of the material of which weld S is constituted, and possibly the permittivity of air if electrodes E1 and E2 are not directly in contact with surfaces 2 and 4.

Therefore, a defect in weld S induces a local change in the materials traversed by field lines LC between the two electrodes E1 and E2, which modifies the local permittivity and, consequently, the measured electrical capacitance.

As illustrated in FIG. 3, the electrical capacitance Cmes is measured for a measurement frequency of between 0 and 1 GHz, for a satisfactorily welded joint (broken line) and for a joint with a weld defect S (continuous line). It is observed that the measured electrical capacitance Cmes has a different value for the satisfactorily welded joint and the poorly welded joint, which enables the method to detect defect S.

Preferentially, the measurement frequency is measured between 100 MHz and 300 MHz. In this frequency range of operation, the electrical impedance measured between these two electrodes is similar as a first approximation to a capacitance.

And, within this range of values, the capacitance difference between a satisfactorily welded joint and a joint presenting a weld defect is particularly detectable, as seen in FIG. 3.

If the measured electrical capacitance Cmes is different by at least 1.5% to the reference value Cref, a defect is detected in weld S.

The reference value is preferably on the order of 5 to 10 pF, preferably from 5 to 7 pF, advantageously on the order of 6 pF, as seen in FIG. 3, for a measurement frequency of between 100 and 300 MHz.

In this case, the method detects a defect in the weld if, during the comparison step, the measured electrical capacitance is different by at least 0.10 pF to the reference value of 6 pF, and preferably different by at least 0.15 pF to the reference value of 6 pF.

Alternatively, the method detects a defect in weld S if the relative difference between the measured capacitance Cmes and the reference value Cref, defined by |Cmes−Cref|/Cref is higher than the uncertainty of measuring the electrical capacitance.

Preferably, the method comprises a step of calibrating the electrical capacitance measurement. In fact, the measured electrical capacitance depends on many parameters, such as the geometry of electrodes E1 and E2, the distance between E1 and E2, the thickness e of conduits, the type of material of the conduits, and the measurement frequency, as will be detailed below. Therefore, the calibration step makes it possible to not make the measurement capacitance Cmes dependent on the quality of the joint.

This step can be experimental by measuring in air or by using reference materials.

Capacitive Probe

Electrodes E1 and E2 are connected to a measuring device capable of measuring electrical capacitance in the frequency range of between 0 and 1 GHz, and particularly between 100 MHz and 300 MHz.

This device can be, for example, an oscillation circuit whose resonance frequency measurement enables the measured electrical capacitance Cmes; a bridge circuit (Wheatstone, Nernst, Sauty, etc.); an impedance meter; and a vector network analyzer, etc., to be deduced.

If for example, the device is an oscillation circuit, electrodes E1 and E2 are connected to it. The resonance frequency measurement fosc enables the measured electrical capacitance Cmes to be deduced.

Electrodes E1 and E2 and the device for measuring the electrical capacitance constitute a capacitive probe.

Electrodes E1 and E2 form a capacitor whose electrical capacitance is the measured electrical capacitance Cmes.

Resonance frequency is determined by the formula

$${f}_{OSC} = \frac{1}{2 π \sqrt{LCmes}} ,$$

where L is the inductance of the oscillation circuit. The measured electrical capacitance Cmes is determined by the formula Cmes=ε0εχ, where ε0 is the absolute permittivity in vacuum, which is on the order of 8.85·10⁻¹² F/m, ε is the relative permittivity of the materials traversed by field lines LC and parameter χ is a factor dependent on the geometry of electrodes (χ=s/D, where s is the surface of electrodes E1 and E2 and D is the distance between electrodes E1 and E2, in a right capacitor model).

As revealed by the previous formulae, the measured capacitance Cmes depends on the geometric characteristics of the electric probe, particularly the surface of electrodes E1 and E2, and the distance D between electrodes E1 and E2.

The measured electrical capacitance also depends on the electrical permittivity ε of the materials traversed, as already explained, this permittivity depending on the measurement frequency.

FIG. 4 illustrates a relative sensitivity of the measured capacitance Cmes compared to the spatial position of an electrical permittivity variation (which can be due to the presence of a defect) according to the present invention depending on the predefined distance D between electrodes E1 and E2. It is observed that the probe can be used in the defect detecting method if the distance D is less than or on the order of the thickness e of conduits 3 and 5, preferably within a range of values between e/5 and e.

It is also noted that the value of the measured electrical capacitance Cmes (the signal) increases with the length of the electrodes while the measurement uncertainty (the noise) of the measured electrical capacitance reduces with the length of the electrodes.

The signal-to-noise ratio increases with electrode length, while the spatial location of a defect S reduces with this length. A good compromise between these two contrary trends is evaluated at a length for electrodes E1 and E2 on the order of the thickness e of conduits 3 and 5, preferably on the order of 50 mm, for example between 45 mm and 55 mm. The electrode geometry can be adjusted according to the minimum size of the investigated defects.

Experimental Result

In the experiment conducted, electrodes E1 and E2 are each constituted of adhesive tape in a substantially rectangular general shape. Their length is on the order of 20 mm and their width is on the order of 10 mm.

Electrode E1 is disposed on the outer surface 2 of the first conduit 3, the direction of the length corresponding to the axial direction of conduits 3 and 5, while the width corresponds to the radial direction of conduits 3 and 5.

Electrodes E1 and E2 are positioned at a distance D on the order of 22 mm.

Electrodes E1 and E2 are connected to a coaxial cable of the RG58 type and a vector network analyzer (of the VNA type, sold under the reference Anritsu 2026C) measures a reflection coefficient S11 on the preferential frequency range of operation, i.e., between 100 MHz and 300 MHz.

Coefficient S11 is determined by the following formula,

$$S 11 = \frac{z - z 0}{z + z 0}$$

where z0 is the impedance of the coaxial cable, on the order of 50Ω, while z is substantially equal to z=−j/Cmesω, j being the complex number, w being the angular velocity, ω=2πf; f is the measurement frequency and Cmes is the measured electrical capacitance.

The electrical capacitance is then measured according to the measurement frequency, between 100 MHz and 300 MHz.

First, the method is applied to an installation in which weld S is of good quality.

Then, the same experiment is repeated on an installation in which the weld S is defective.

The experimental results obtained are illustrated in FIGS. 5 and 6.

As revealed by FIG. 5, the measured capacitance Cmes is on the order of 6 pF between 50 MHz and 300 MHz.

The measured capacitance difference between the installation with a good weld and a defective weld is on the order of 0.15 pF, which represents a relative capacitance difference on the order of 2.5%.

FIG. 6 illustrates the evolution in the value of the relative capacitance difference according to measurement frequency, for a measurement frequency of between 50 and 300 MHz.

The uncertainty of measuring electrical capacitance in this experiment is evaluated at 1.5% by measuring the repeatability of the experiment.

Therefore, as the relative capacitance difference is higher than the measurement uncertainty, the weld defect is detected by the method according to the present invention.

Embodiment of the Method for Detecting Defects in an Installation of HDPE Conduits with Winding

FIGS. 7 and 8 illustrate another embodiment of an installation of HDPE conduits equipped with electrical wire windings.

Installation 1 comprises a first conduit 3 and a second conduit 5 in HDPE. Conduits 3 and 5 are joined by their ends in a joining region 1 and present a general coaxial cylinder shape.

Installation 1 comprises a sleeve 7 in HDPE, which envelopes conduits 3 and 5 in the joining region 1.

Two electrical wire windings 8, 9, are wound on sleeve 7, on both sides of joining region 1 between conduits 3 and 5. Sleeve 7 is welded to conduits 3 and 5, for example upon heating the windings powered by a power source.

The defect detection method here applies to the detection of the defect of weld S between sleeve 7 and conduit 3 or conduit 5.

According to a first variation illustrated in FIG. 7, electrode E1 is constituted of winding 8, electrode E2 being placed on conduit 3. Field lines LC pass through weld S.

In this variation, the first surface is sleeve 7 while the second surface is the periphery surface 2 of conduit 3.

The method enables a defect in weld S between sleeve 7 and conduit 3 to be detected. A defect in weld S between sleeve 7 and conduit 5 can also be detected if electrode E2 is placed on conduit 5.

According to a second variation illustrated in FIG. 8, electrode E1 is placed on sleeve 7 close to winding 8 (being distinct from winding 8), electrode E2 being disposed on conduit 3, as in FIG. 8. Field lines LC traverse weld S.

The method enables a defect in weld S between sleeve 7 and conduit 3 to be detected. A defect in weld S between sleeve 7 and conduit 5 can also be detected if electrode E1 is placed on sleeve 7 close to winding 9 and if electrode E2 is placed on conduit 5.

Windings 8, 9 are illustrated as being distinct but they, of course, can form part of a single winding.

Such an installation 1 is found for example in natural gas or water systems.

Utilization

Another aim of the invention is a use of a capacitive probe as described previously for detecting a defect in a weld joining a first surface of a polyethylene (PE)-based material, advantageously in high-density polyethylene and a second surface of a polyethylene (PE)-based material, advantageously in high-density polyethylene.

Advantageously, the first surface is coaxial and adjacent to the second surface. This is the embodiment illustrated in FIGS. 1 to 6.

Alternatively, the first surface is coaxial and at least partly fitted to the second surface. This is the embodiment illustrated in FIGS. 7 and 8.

1. A method of detecting a defect in a weld joining a first surface of a polyethylene (PE)-based material and a second surface of a polyethylene (PE)-based material, said method comprising the steps of:
positioning a first electrode and a second electrode in proximity to the weld, so that at least one electric field line passes through said weld when a potential difference is applied between said first and second electrodes,
measuring an electrical capacitance at a measurement frequency between said first electrode and said second electrode, the measurement frequency being higher than 65 MHz and lower than 1 GHz in order to compare the measured electrical capacitance to a reference value.

2. The method according to claim 1, wherein the measurement frequency is measured between 100 MHz and 300 MHz.

3. The method according to claim 2, comprising a step of detecting a defect in the weld if, during the comparison step, the measured electrical capacitance is different by at least 1.5% from the reference value.

4. The method according to claim 3, wherein the reference value is on the order of 6 pF.

5. The method according to claim 1, wherein the first surface belongs to a body presenting one thickness and the second surface belongs to a body presenting a thickness of the same order of magnitude as the thickness of the body of the first surface, and in which the predefined distance between the first electrode and the second electrode is within a range of values between a fifth of the value of said thickness and the value of said thickness.

6. A capacitive probe for implementing the method according to claim 1, comprising a first electrode and a second electrode forming a capacitor presenting an electrical capacitance capable of depending on the presence of a defect in a weld joining a first surface of a polyethylene (PE)-based material and a second surface of a polyethylene (PE)-based material, said probe comprising a measuring device capable of measuring an electrical capacitance within a measurement frequency of between 65 MHz and 1 GHz.

7. The probe according to claim 6, wherein said measuring device is an oscillation circuit, the resonance frequency measurement of which enables the measured electrical capacitance or a bridge circuit or an impedance meter or a vector network analyzer to be deduced.

8. The probe according to claim 6, wherein the measurement frequency is between 100 MHz and 300 MHz.

9. The probe according to claim 6, wherein the first electrode and the second electrode have a length on the order of a thickness of a body comprising said first surface and a body comprising said second surface.

10. The probe according to claim 6, wherein the first electrode is constituted of a winding of electrical wires.

11. The utilization of a capacitive probe according to claim 6 for detecting a defect in a weld joining a first surface of a polyethylene (PE)-based material and a second surface of a polyethylene (PE)-based material.

12. The utilization according to claim 11, wherein the first surface is coaxial and adjacent to the second surface.

13. The utilization according to claim 11, wherein the first surface is coaxial and at least partly fitted to the second surface.
